(19) United States
(12) Patent Application Publication
(10) Pub. No.: US 2023/0310427 A1
(43) Pub. Date: Oct. 5, 2023

(54) NEW THERAPEUTIC USE OF RILPIVIRINE

(71) Applicant: UNIVERSITY OF SOUTH AUSTRALIA, Adelaide, South Australia (AU)

(72) Inventors: Richard John HEAD, Seaview Downs (AU); Shudong WANG, Adelaide (AU); Md Saiful ISLAM, Klemzig (AU); Malika KUMARASIRI, Oxley, Queensland (AU)

(21) Appl. No.: 18/003,733

(22) PCT Filed: Jun. 30, 2021
(86) PCT No.: PCT/AU2021/000045

§ 371 Date: Dec. 29, 2022

(30) Foreign Application Priority Data
Jun. 30, 2020 (AU) 2020902207

(51) Int. Cl. A61K 31/505; A61P 35/00; A61K 31/337; A61P 15/08; A61P 13/08; A61P 35/02

(52) U.S. Cl. CPC A61K 31/505; A61K 31/337; A61P 13/08; A61P 15/08; A61P 35/00; A61P 35/02

(57) ABSTRACT

Methods and uses of Rilpivirine and analogues thereof in the treatment and/or prevention of proliferative cell diseases and conditions are disclosed. Among the diseases and conditions that may be treated and/or prevented, are cancers characterised by Aurora A dysregulation.

### TECHNICAL FIELD

The present disclosure relates to a novel use of Rilpivirine and analogues thereof in the treatment and/or prevention of proliferative cell diseases and conditions including cancers.

### PRIORITY DOCUMENT

The present application claims priority from Australian Provisional Patent Application No 2020902207 titled “New therapeutic use of rilpivirine” and filed on 30 Jun. 2020, the content of which is hereby incorporated by reference in its entirety.

### BACKGROUND

Protein kinases regulate various biological functions, including DNA replication, transcription, translation, cell cycle progression, energy metabolism, migration and cell growth, making them excellent targets for treating proliferative diseases and conditions including cancers. The identification of compounds which inhibit or modulate the activity of protein kinases and which may be developed into effective therapeutic anti-proliferative agents, are still needed.

Rilpivirine (ie 4-[[4-[4-[(E)-2-cyanoethenyl]-2,6-dimethylanilino]pyrimidin-2-yl]amino]benzonitrile) is an anti-viral drug compound particularly developed for the treatment of patients suffering from an infection by the Human Immunodeficiency Virus (HIV). The compound belongs to the class of anti-viral drug compounds known as non-nucleoside reverse transcriptase inhibitors (NNRTIs) and was approved for use in anti-HIV drug cocktails in the United States in May 2011 under the brand name Edurant® (Janssen Therapeutics, Titusville, NJ, United States of America). It is considered to inhibit the ability of HIV to reproduce by inhibiting the viral enzyme, reverse transcriptase, so as to prevent synthesis of the double-stranded viral DNA required for integration into the host cell’s genome and subsequent reproduction of the virus. The synthesis of this compound and its utility in the treatment of HIV infection has been described in the International Patent Publication No WO 03/016306 (the entire disclosure of which is to be regarded as incorporated herein by reference).

### SUMMARY

Surprisingly, it has been found by the present applicant that the anti-viral drug compound Rilpivirine (and analogues thereof), also has utility in the treatment and/or prevention of cancers and other proliferative cell diseases and conditions.

Thus, in a first aspect, the present disclosure provides a method of treating and/or preventing cancer or another proliferative cell disease or condition in a subject, the method comprising administering to said subject a therapeutically effective amount of Rilpivirine or an analogue thereof, or a pharmaceutically acceptable salt, solvate or prodrug thereof, optionally in combination with a pharmaceutically acceptable carrier, diluent and/or excipient.

In some preferred embodiments, the method of the first aspect is used to treat and/or prevent cancers selected from leukaemia, ovarian, lung, colorectal, cervical, neural, breast, prostate and melanoma cancers, and preferably those characterised by dysregulation of protein kinases, especially Aurora A.

In some embodiments, the Rilpivirine (or analogue thereof) may be administered in combination with one or more additional agent(s) for the treatment of cancer or another proliferative disease or condition. For example, the compound may be used in combination with other anti-cancer agents in order to inhibit more than one cancer signalling pathway simultaneously so as to make cancer cells more susceptible to anti-cancer therapies (eg treatments with other anti-cancer agents, chemotherapy, radiotherapy or a combination thereof).

In a second aspect, the present disclosure provides the use of Rilpivirine or an analogue thereof, or a pharmaceutically acceptable salt, solvate or prodrug thereof, for treating and/or preventing cancer or another proliferative cell disease or condition.

In a third aspect, the present disclosure provides the use of Rilpivirine or an analogue thereof, or a pharmaceutically acceptable salt, solvate or prodrug thereof, in the manufacture of a medicament (such as a pharmaceutical composition) for treating and/or preventing cancer or another proliferative cell disease or condition.

In a fourth aspect, the present disclosure provides a pharmaceutical composition comprising Rilpivirine or an analogue thereof, or a pharmaceutically acceptable salt, solvate or prodrug thereof, and a pharmaceutically acceptable carrier, diluent and/or excipient for use in treating and/or preventing cancer or another proliferative cell disease or condition.

In a fifth aspect, the present disclosure provides a method for modulating Aurora A kinase activity in a cell, comprising introducing to or contacting said cell with an effective amount of Rilpivirine or an analogue thereof, or a pharmaceutically acceptable salt, solvate or prodrug thereof.

### BRIEF DESCRIPTION OF FIGURES

FIG. 1 shows the effect of Rilpivirine on proliferation of Leukemia (HL-60, U-937, NB4, Jurkat, K-562), ovarian (A2780), prostate (PC3) and breast (T47D) cancer cell lines following 72 hour treatment using cell viability assays. The results indicate that Rilpivirine inhibits the proliferation of different cancer cell lines with an IC₅₀ value ranging from 3 to 8 µM;

FIG. 2 shows kinase inhibition profiling of Rilpivirine and Etravirine. (A) Inhibition (% inhibition) of kinase activity was determined for a panel of 45 kinases at 10 µM Rilpivirine. (B) Inhibition constant (Kᵢ) values of Rilpivirine and Etravirine were also determined against Aurora A and Aurora B kinase. The results demonstrate that Rilpivirine potently inhibited Aurora A kinase and also has inhibitory activity against Aurora B, PIM1, JAK1 and FLT3 kinases, but Etravirine has no inhibitory activity on Aurora A or Aurora B kinase;

FIG. 3 provides the results of flow cytometric analysis of the leukaemia cell line HL-60 following a 24 hour treatment with diluent only or 5, 10 and 20 µM Rilpivirine; the results show that treatment with the compound arrests the cell in the G2/M phase;

FIG. 4 provides the results of flow cytometric analysis of the breast cancer cell line T47D following a 48-hour treatment with Rilpivirine (Ril) at various concentrations, or the comparator drugs Alisertib (Ali) and Etravirine (Etra). The results demonstrated that Rilpivirine and Alisertib arrest T47D breast cancer cells in G2/M phase, but no G2/M arrest was evident for Etravirine at 10 µM;

FIG. 5 provides the results of flow cytometric analysis of the prostate cancer cell line PC3 following a 48 hour treatment with Rilpivirine (Ril) at various concentrations, or the comparator drugs Alisertib (Ali) and Etravirine (Etra). The results showed that Rilpivirine and Alisertib induce G2/M arrest in PC3 cells but Etravirine did not induce G2/M arrest at 10 µM;

FIG. 6 provides the results of flow cytometric analysis of the leukaemic NB4 and Jurkat cell lines following treatment with Rilpivirine at various concentrations for a 24 hour period; the results demonstrated that Rilpivirine arrests NB4 and Jurkat cells in the G2/M phase in a dose-dependent manner with sub-G 1 content indicating apoptosis;

FIG. 7 provides the results of flow cytometric analysis of the leukaemic K-562 and U-937 cell lines following treatment with Rilpivirine at various concentrations for a 24 hour period; the results demonstrated that Rilpivirine arrests both of these cell lines in the G2/M phase in a dose-dependent manner with sub-G1 content indicating apoptosis;

FIG. 8 shows the results of apoptosis analysis of HL-60 (A), and NB4 and Jurkat (B) cells following a 48 hour treatment with Rilpivirine at the various concentrations indicated. Apoptosis was assessed using FITC-conjugated annexinV and propidium iodide (PI) and subjected to flow cytometric analysis. In each plot, the Lower Left (LL) shows live cells; the Lower Right (LR) shows early apoptotic cells; the Upper Right (UR) shows late apoptotic cells; and the Upper Left (UL) shows necrotic cells. The results demonstrated that Rilpivirine induces apoptosis in all of these cell lines in a dose-dependent manner;

FIG. 9 shows an image of Western blot obtained from cell lysates of HL-60 and NB4 cells following 24 hour treatments with Rilpivirine at various concentrations. The blots clearly show that there is a decrease in the amount of auto-phosphorylation of Aurora A with increasing amounts of Rilpivirine;

FIG. 10 provides the results of flow cytometric analysis of p53 wild-type (WT) A2780 cells after 24, 48 and 72 hour treatments with 5 µM, 10 µM and 20 µM Rilpivirine; the results show that treatment with the compound arrests the cells in the G1 phase at 10 µM concentration but at 20 µM, increased number of cells accumulated in Sub-G1 phase;

FIG. 11 provides the results of flow cytometric analysis of the p53 WT ovarian cell line, OVCAR-5, after 72 hour treatments with 5 µM, 10 µM and 20 µM Rilpivirine; the results show that treatment with the compound arrests the cells in the G1 phase or accumulated in Sub-G1 phase at higher concentrations;

FIG. 12 shows an image of a Western blot obtained from cell lysates of p53 WT A2780 cells following 24 hour treatments with Rilpivirine at various concentrations. The blot clearly shows that there is a decrease in the amount of phosphorylated retinoblastoma (Rb) protein and cyclin D1 (known to associate with G1-S phase transition of cell cycle; Landriscina M et al., Curr Pharm Des 13:737-747, 2007) with increasing amounts of Rilpivirine. Increasing concentration of Rilpivirine also brought about an increase in the expression of p53 and induced its target gene p21, which itself is an inhibitor for G1-S phase transition;

FIG. 13 provides the results of assays to determine the effect of Rilpivirine on p53 and cyclin D1 mRNA transcription following 24 hour treatments of p53 WT A2780 cells;

FIG. 14 shows the results of apoptosis analysis of p53 WT A2780 cells following 48 hour treatments with Rilpivirine at various concentrations indicated. The results demonstrated that Rilpivirine induces apoptosis in this cell line in a dose-dependent manner;

FIG. 15 shows an image of a Western blot obtained from cell lysates of p53 WT A2780 cells following 48 hour treatments with Rilpivirine at various concentrations. The blot clearly shows that apoptosis is associated with cleavage of PARP (poly (ADP-ribose) polymerase); and

FIG. 16 shows the effect of Rilpivirine in combination with Docetaxel in the MDA-MB-453 cell line. MDA-MB-453 cells were seeded in 96-wellplates and treated with either single agent or varying concentrations of combinations of Rilpivirine and Docetaxel for 72 h. (A) Dose response curve of MDA-MB-453 cells exposed to Rilpivirine and Docetaxel as single agent; (B) Growth relative to diluent control is shown in plot, with the first column and first row treated with the single agent Rilpivirine and Docetaxel respectively. All other wells have varying concentrations of the two agents in combination; (C) Plot shows the predictions made by the Bliss mathematical model; and (D) Plot shows the difference between the model prediction and experimental data. The positive synergy score >25 in plot (D) clearly identifies the region of synergy.

### DETAILED DESCRIPTION

The present applicant has found that Rilpivirine and analogues thereof are suitable for use in the prevention and/or treatment of proliferative cell diseases and conditions including cancers. More particularly, it has been found that Rilpivirine and analogues thereof are capable of potently inhibiting the Aurora A kinase (ie using a cell free kinase assay, it was found that Rilpivirine in particular, potently inhibited Aurora A with a Kᵢ value of 0.13 (µM)), which is a serine/threonine kinase that is believed to have an important role(s) during cell mitosis and shows peak activity during the G2 phase to M phase transition of the cell cycle (Hannak E et al., J Cell Biol 155(7):1109-1116, 2001; Marumoto T et al., J Biol Chem 278(51):51786-51795, 2003). Aurora A dysregulation (eg the overexpression of Aurora A and/or amplification of the gene encoding Aurora A) has been linked to a number of proliferative cell diseases and conditions including, for example, some ovarian, lung, colorectal, cervical, neural, breast, prostate and melanoma cancers, as well as leukaemias (Yan M et al., Med Res Rev 36(6): 1036-1079, 2016), and some Aurora A inhibitors have been identified and/or developed for treating some of these cancers including lung cancer (Chinn DC et al., J Cancer Res Clin Oncol 140(7):1137-1349, 2014), prostate cancer (Paller CJ et al., Cancer Med 3(5):1322-1335, 2014), and neuroblastoma (Michaelis et al., PLoS One 9(9): e108758, 2014). As such, Rilpivirine and analogues thereof may be particularly suitable for treating and/or preventing cancers and other proliferative cell diseases and conditions characterised by kinase dysregulation (eg kinase overexpression and/or gene amplification), including those which are characterised by dysregulation of one or more of Aurora A, Aurora B, PIM1 (Proto-oncogene serine/threonine-protein kinase-1), JAK1 (Janus kinase 1), FLT3 (fms-like tyrosine kinase 3),YES and LYN (Src family of tyrosine kinases).

Thus, in a first aspect, the present disclosure provides a method of treating and/or preventing cancer or another proliferative cell disease or condition in a subject, the method comprising administering to said subject a therapeutically effective amount of Rilpivirine or an analogue thereof, or a pharmaceutically acceptable salt, solvate or prodrug thereof, optionally in combination with a pharmaceutically acceptable carrier, diluent and/or excipient.

In a second aspect, the present disclosure provides the use of Rilpivirine or an analogue thereof, or a pharmaceutically acceptable salt, solvate or prodrug thereof, for treating and/or preventing cancer or another proliferative cell disease or condition.

In a third aspect, the present disclosure provides the use of Rilpivirine or an analogue thereof, or a pharmaceutically acceptable salt, solvate or prodrug thereof, in the manufacture of a medicament (such as a pharmaceutical composition) for treating and/or preventing cancer or another proliferative cell disease or condition.

In a fourth aspect, the present disclosure provides a pharmaceutical composition comprising Rilpivirine or an analogue thereof, or a pharmaceutically acceptable salt, solvate or prodrug thereof, and a pharmaceutically acceptable carrier, diluent and/or excipient for use in treating and/or preventing cancer or another proliferative cell disease or condition.

In a fifth aspect, the present disclosure provides a method for modulating Aurora A kinase activity in a cell, comprising introducing to or contacting said cell with an effective amount of Rilpivirine or an analogue thereof, or a pharmaceutically acceptable salt, solvate or prodrug thereof.

As mentioned above, Rilpivirine is an anti-viral drug compound known as 4-[[4-[4-[(E)-2-cyanoethenyl]-2,6-dimethylanilino]pyrimidin-2-yl]amino]benzonitrile and having the structure:

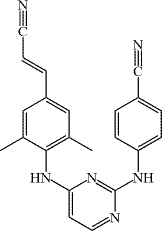 [SMILES: Cc1cc(/C=C/C#N)cc(C)c1Nc1ccnc(Nc2ccc(C#N)cc2)n1]

For the purposes of the present specification, it is to be understood that analogues of Rilpivirine comprise compounds (which are not Rilpivirine) according to the following formula (I):

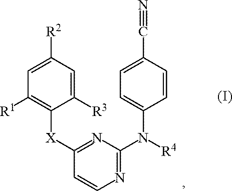

wherein:
R¹, R³ and R⁴ are each independently selected from the group consisting of H, saturated and unsaturated, optionally substituted, aliphatic hydrocarbons such as alkyl (eg a C₁₋₆ alkyl or, preferably, a C₁₋₃ alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne), halogen (especially Br or F), NO₂, CF₃, OH, optionally substituted O-alkyl (eg an O-C₁₋₆ alkyl, preferably, an O-C₁₋₃ alkyl such as O—CH₃), NH₂, optionally substituted NH-alkyl (eg a NH-C₁₋₆ alkyl, preferably, a NH-C₁₋₃ alkyl such as NH—CH₃), N(alkyl)₂ (such as N(CH₃)₂), and optionally substituted SH-alkyl (eg a SH-C₁₋₆ alkyl or, preferably, a SH-C₁₋₃ alkyl such as SHCH₃ and SHC(CH₃));
R² is selected from the group consisting of saturated and unsaturated aliphatic hydrocarbons such as alkyl (eg a C₁₋₆ alkyl or, preferably, a C₁₋₃alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne) substituted with one or more substituents each independently selected from CN, NR⁵R⁶, —C(═O)—NR⁵R⁶ and —C(═O)—C₁₋₆ alkyl; wherein R⁵and R⁶ are each independently selected from H, OH, C₁₋₆alkyl (preferably, a C₁₋₃alkyl such as methyl), C₁₋₆alkyloxy (preferably, a C₁₋₃ alkyloxy), C₁₋₆alkylcarbonyl (preferably, a C₁₋₃ alkylcarbonyl), C₁₋₆alkyloxycarbonyl (preferably, a C₁₋₃ alkyloxycarbonyl), NH₂, and mono- or di-(C₁₋₆alkyl)amino; and
X is selected from NH, N(C₁₋₃alkyl), O, S, CH₂ and C(CH₃)_{2;}
or a pharmaceutically acceptable salt, solvate or prodrug thereof.

In some embodiments, R¹ and R³ of the Rilpivirine analogues are each independently selected from H and optionally substituted C₁₋₆alkyl (preferably, C₁₋₃ alkyl such as methyl).

In some preferred embodiments, R¹ and R³ of the Rilpivirine analogues are each independently selected from C₁₋₃alkyl.

In some embodiments, R⁴ of the Rilpivirine analogues is selected from H, formyl, C₁₋₆ alkyl (preferably, a C₁₋₃alkyl such as methyl), C₁₋₆ alkyl (preferably, a C₁₋₃alkyl) substituted with formyl, C₁₋₆ alkylcarbonyl (preferably, a C₁₋₃ alkylcarbonyl), C₁₋₆alkyloxycarbonyl (preferably, a C₁₋₃ alkyloxycarbonyl), C₁₋₆alkylcarbonyloxy (preferably, a C₁₋₃alkylcarbonyloxy), and C₁₋₆ alkyloxy-C₁₋₆ alkylcarbonyl substituted with C₁₋₆alkyloxycarbonyl (preferably, a C₁₋₃ alkylcarbonyloxy).

In some preferred embodiments, R⁴ of the Rilpivirine analogues is selected from H and C₁₋₆alkyl (preferably, a C₁₋₃alkyl such as methyl).

In some embodiments, R² is selected from saturated and unsaturated linear aliphatic hydrocarbons such as alkyl (eg a C₁₋₆ alkyl or, preferably, a C₁₋₃alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne) substituted with one or more substituents each independently selected from CN, NR⁵R⁶, —C(═O)—NR⁵R⁶ and -C(=O)-C₁₋₆ alkyl.

In some preferred embodiments, R² is selected from ethyl or ethene substituted with CN, NR⁵R⁶, —C(═O)— NR⁵R⁶ and -C(=O)-C₁₋₆alkyl.

In some preferred embodiments, X of the Rilpivirine analogues is NH.

In this specification, a number of terms are used which are well known to those skilled in the art. Nevertheless, for the purposes of clarity, a number of these terms are hereinafter defined.

As used herein, the term “aliphatic” takes its normal meaning in the art and includes non-aromatic hydrocarbon groups such as alkanes (alkyl), alkenes and alkynes and substituted derivatives thereof.

As used herein, the term “alkyl” includes both linear (ie straight) chain and branched alkyl groups having from 1 to 8 carbon atoms (eg methyl, ethyl, propyl, isopropyl, butyl, isobutyl, tert-butyl, pentyl, hexyl etc).

The term “halogen” refers to fluoro, chloro, bromo and iodo.

As used herein, the term “optionally substituted” means that the relevant component of the Rilpivirine analogues of formula (I), for example, the “aliphatic hydrocarbon” option for R¹, R³ and R⁴ may or may not be substituted with one or more substituent group(s) such as those well known to those skilled in the art including, for example, OH, halogen, NH₂, carbonyl, C₁₋₆alkyl, C₁₋₆ alkylamino etc.

As used herein, the phrase “manufacture of a medicament” includes the use of one or more of Rilpivirine and analogues thereof directly as the medicament or in any stage of the manufacture of a medicament (such as a pharmaceutical composition) comprising one or more of Rilpivirine and analogues thereof.

Some of the Rilpivirine analogues of formula (I) may exist as single stereoisomers, racemates, and/or mixtures of enantiomers and /or diastereomers. All such single stereoisomers, racemates and mixtures thereof, are encompassed within the scope of the present disclosure. The isomeric forms such as diastereomers, enantiomers, and geometrical isomers can be separated by physical and/or chemical methods well known to those skilled in the art.

The term “pharmaceutically acceptable salt” as used herein, refers to salts that retain the desired biological activity of the compounds of Formula I, and include pharmaceutically acceptable acid addition salts and base addition salts. Suitable pharmaceutically acceptable acid addition salts of the compounds of Formula I may be prepared from an inorganic acid or from an organic acid. Examples of such inorganic acids are hydrochloric, sulfuric and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic. Additional information on pharmaceutically acceptable salts can be found in Remington’s Pharmaceutical Sciences, 19th Edition, Mack Publishing Co., Easton, PA 1995.

The term “solvate” refers to any form of the compounds of Formula I, resulting from solvation of with an appropriate solvent. Such a form may be, for example, a crystalline solvate or a complex that maybe formed between the solvent and the dissolved compound.

The term “prodrug” means a compound that undergoes conversion to a compound of Formula I within a biological system, usually by metabolic means (eg by hydrolysis, reduction or oxidation). For example, an ester prodrug of a compound of Formula I containing a hydroxyl group may be convertible by hydrolysis in vivo to the compound of Formula I. Suitable esters of the compounds of Formula I containing a hydroxyl group may be, for example, acetates, citrates, lactates, tartrates, malonates, oxalates, salicylates, propionates, succinates, fumarates, maleates, methylene-bis-P-hydroxynaphthoates, gestisates, isethionates, di-p-toluoyltartrates, methanesulfonates, ethanesulfonates, benzenesulfonates, p-toluenesulfonates, cyclohexylsulfamates and quinates. As another example, an ester prodrug of a compound of Formula I containing a carboxy group may be convertible by hydrolysis in vivo to the compound of Formula I. Examples of ester prodrugs include those described by Leinweber FJ, Drug Metab Rev 18:379-439 (1987). Similarly, an acyl prodrug of a compound of Formula I containing an amino group may be convertible by hydrolysis in vivo to the compound of Formula I. Examples of prodrugs for these and other functional groups, including amines, are provided in Prodrugs: challenges and rewards, Valentino J Stella (ed), Springer, 2007.

In some preferred embodiments, the methods, uses and composition of the present disclosure employ a hydrochloride salt(s) of the Rilpivirine or an analogue thereof.

In the case of Rilpivirine or analogues of formula (I) that are solid, it will be understood by those skilled in the art that the compounds (or pharmaceutically acceptable salts, solvates or prodrugs thereof) may exist in different crystalline or polymorphic forms, all of which are encompassed within the scope of the present disclosure.

The term “therapeutically effective amount” or “effective amount” is an amount sufficient to effect beneficial clinical or desired results. A therapeutically effective amount can be administered in one or more administrations. Typically, a therapeutically effective amount is sufficient for treating and/or preventing a disease or condition or otherwise to palliate, ameliorate, stabilise, reverse, slow or delay the progression of a cancer or another proliferative cell disease or condition. By way of example only, a therapeutically effective amount of Rilpivirine or an analogue of formula (I), or a pharmaceutically acceptable salt, solvate or prodrug thereof, may comprise between about 0.1 and about 500 mg/kg body weight per day, such as, for example, between about 0.1 and about 25 mg/kg body weight per day, or between about 0.1 and about 450 mg/kg body weight per day, or between about 150 to about 450 mg/kg body weight per day, and in some embodiments, the therapeutically effective amount will be an amount greater than about 25 mg/kg body weight per day. However, notwithstanding the above, it will be understood by those skilled in the art that the therapeutically effective amount may vary and depend upon a variety of factors including the activity of the particular compound (or salt, solvate or prodrug thereof), the metabolic stability and length of action of the particular compound (or salt, solvate or prodrug thereof), the age, body weight, sex, health, route and time of administration, rate of excretion of the particular compound (or salt, solvate or prodrug thereof), and the severity of, for example, the cancer or other proliferative cell disease or condition to be treated.

Rilpivirine and the analogues of formula (I) are inhibitors of Aurora A kinase and are therefore considered to be particularly well suited for use in methods and applications for the treatment and/or prevention of cancer and other proliferative cell diseases and conditions characterised by Aurora A dysregulation (eg the overexpression or amplification of Aurora A). Whether a subject’s particular cancer or other proliferative disease or condition is or is not characterised by Aurora A dysregulation can be readily determined by assaying a cell lysate from a suitable tissue sample (eg a blood sample or biopsy) and quantitatively or qualitatively assessing the level of Aurora A (or Aurora A mRNA) present using any of the methods well known to those skilled in the art (including, for example, Western blot and quantitative amplification techniques such as qPCR). Those skilled in the art will be able to readily identify from such assay results whether the subject’s cancer or other proliferative disease or condition is or is not characterised by Aurora A dysregulation.

Aurora A dysregulation has been linked to a number of proliferative cell diseases and conditions including, for example, solid cancers such as some ovarian, lung, colorectal, cervical, neural, breast, prostate and melanoma cancers, and non-solid cancers such as leukaemias.

Thus, in some preferred embodiments, the methods, uses and composition of the present disclosure are used to treat and/or prevent cancers selected from haematologic malignancies (eg leukaemias) and ovarian, lung, colorectal, cervical, neural, breast, prostate and melanoma cancers, and preferably those characterised by Aurora A dysregulation.

In some embodiments, the methods, uses and composition of the present disclosure are used to treat and/or prevent cancers selected from haematologic malignancies (eg leukaemias) and ovarian, lung, colorectal, cervical, neural, breast, prostate and melanoma cancers, and preferably those characterised by Aurora A dysregulation, but excluding cancers related to HIV infection (known as “HIV infection cancer” or “HIV-associated cancers”).

In some embodiments, the methods, uses and composition of the present disclosure are used to treat and/or prevent a proliferative cell disease or condition associated with some cardiovascular diseases or conditions such as restenosis and cardiomyopathy, some auto-immune diseases such as glomerulonephritis and rheumatoid arthritis, dermatological conditions such as psoriasis, and fungal or parasitic disorders.

Rilpivirine and the analogues of formula (I) may exert an anti-proliferative effect and/or induce apoptosis by acting at a cell cycle “checkpoint”. In particular, Rilpivirine (and analogues thereof) may act at the G1 and/or G2/M phase to cause cell cycle arrest. Surprisingly, it has been found that Rilpivirine (and analogues thereof) will show an increased propensity to arrest a cell at the G1 phase or G2/M phase depending upon the “p53 status” of the cell. The p53 protein is well known to those skilled in the art; it is a key tumour suppressor protein and has been well characterised in the context of cancer, where it is a common point of mutation. Amongst other things, it is considered that the p53 protein may associate with cyclin D1 in G1 arrest. When cells treated with Rilpivirine (or analogues thereof) are “p53 wild-type” (ie the cell expresses functional p53 protein; otherwise denoted as “p53 positive”), the cells are arrested predominantly in the G1 phase, whereas when the cells are “p53 negative” (ie the cell does not express p53 (“null”) or does not express a functional p53 (“mutant”)), treatment with Rilpivirine (or analogues thereof) are arrested predominantly in the G2/M phase. This may have important implications in the treatment and/or prevention of cancers and other proliferative diseases and conditions. That is, the successful treatment and/or prevention of cancers and other proliferative diseases and conditions with Rilpivirine or an analogue thereof may be independent of p53 status, meaning that successful outcomes may be achieved with a broad range of cancers and other proliferative diseases and conditions (eg both p53 wild-type and p53 negative cancers). This may also mean that there is no need to characterise the disease or condition to be treated for p53 status; although this knowledge may also inform further treatment design or choice (eg p53 status may assist in the selection of other drug compounds (eg other anti-cancer agents) in combination therapies with Rilpivirine or an analogue thereof). Further, Rilpivirine (and analogues thereof) may be less likely to lead to drug resistance (ie through mutation), since there are two potential checkpoints targeted by the drug.

In accordance with the present disclosure, Rilpivirine and analogues thereof may be used for both treating and/or preventing cancers and other proliferative cell diseases and conditions. As such, it is to be appreciated that the scope of the present disclosure includes prophylaxis as well as the alleviation of established symptoms of the cancer or other proliferative cell disease or condition. As such, the methods and uses of Rilpivirine and analogues thereof in accordance with the present disclosure includes: (1) preventing or delaying the appearance of clinical symptoms of the cancer or other proliferative disease or condition developing in a subject afflicted with or predisposed to the cancer or other proliferative disease or condition; (2) inhibiting the cancer or other proliferative disease or condition (ie arresting, reducing or delaying the development of the cancer or other proliferative disease or condition) or a relapse thereof (in case of a maintenance treatment) or at least one clinical or subclinical symptom thereof; and (3) relieving or attenuating the cancer or other proliferative disease or condition (ie causing regression of the cancer or other proliferative disease or condition or at least one of its clinical or subclinical symptoms).

In some preferred embodiments, the Rilpivirine (or an analogue thereof) used in the methods, uses and composition of the present disclosure, exhibits anti-proliferative activity in human cell lines, as measured by a standard cytotoxicity assay. Preferably, the compound exhibits an IC₅₀ value of less than 10 µM. More preferably still, the compound exhibits an IC₅₀ value of less than 5 µM.

In some preferred embodiments, the Rilpivirine (or an analogue thereof) used in the methods, uses and composition of the present disclosure, inhibits Aurora A kinase, as measured by any standard assay well known to those skilled in the art. Preferably, the Rilpivirine (or analogue thereof) exhibits an IC₅₀ value of less than 1 µM, more preferably less than 0.5 µM.

Rilpivirine (or analogues thereof) may be administered in combination with one or more additional agent(s) for the treatment of cancer or another proliferative disease or condition. For example, the compounds may be used in combination with other anti-cancer agents in order to inhibit more than one cancer signalling pathway simultaneously so as to make cancer cells more susceptible to anti-cancer therapies (eg treatments with other anti-cancer agents, chemotherapy, radiotherapy or a combination thereof). As such, Rilpivirine or the analogues of formula (I) may be used in combination with one or more of the following categories of anti-cancer agents:
anti-proliferative/antineoplastic drugs such as alkylating agents (eg cis-platin, oxaliplatin, carboplatin, cyclophosphamide, nitrogen mustard, melphalan, chlorambucil, busulphan, temozolamide and nitrosoureas); antimetabolites (eg gemcitabine and antifolates such as fluoropyrimidines like 5-fluorouracil and tegafur, raltitrexed, methotrexate, cytosine arabinoside, fludarabine and hydroxyurea); antitumour antibiotics (eg anthracyclines such as adriamycin, bleomycin, doxorubicin, daunomycin, epirubicin, idarubicin, mitomycin-C, dactinomycin and mithramycin); antimitotic agents (eg vinca alkaloids such as vincristine, vinblastine, vindesine and vinorelbine and taxanes including docetaxel (taxotere) and paclitaxel (taxol) (Falchook G et al., JAMA Oncol 5(1):e183773, 2019) and polokinase inhibitors); TORC½ inhibitors such as sapanisertib (TAK-228); and topoisomerase inhibitors (eg pipodophyllotoxins such as etoposide and teniposide, amsacrine, topotecan and camptothecin);
cytostatic agents such as antioestrogens (eg tamoxifen, fulvestrant, toremifene, raloxifene, droloxifene and iodoxyfene), antiandrogens (eg bicalutamide, flutamide, nilutamide and cyproterone acetate), LHRH antagonists or LHRH agonists (eg goserelin, leuprorelin and buserelin), progestogens (eg megestrol acetate), aromatase inhibitors (eg as anastrozole, letrozole, vorazole and exemestane) and inhibitors of 5α-reductase such as finasteride;
anti-invasion agents (eg c-Src kinase family inhibitors such as 4-(6-chloro-2,3-methylenedioxyanilino)-7-[2-(4-methylpiperazin-1-yl)ethoxy]-5-tetrahydropyran-4-yloxyquinazoline (AZD0530; International Patent Publication No WO 01/94341), N-(2-chloro-6-methylphenyl)-2- {6-[4-(2-hydroxyethyl)piperazin-1-yl]-2-methylpyrimidin-4-ylamino}thiazole-5-carboxamide (dasatinib) and bosutinib (SKI-606)), and metalloproteinase inhibitors including marimastat, inhibitors of urokinase plasminogen activator receptor function or antibodies to heparanase;
inhibitors of growth factor function (eg growth factor antibodies and growth factor receptor antibodies such as the anti-erbB2 antibody trastuzumab (Herceptin™), the anti-EGFR antibody panitumumab, the anti-erbBl antibody cetuximab (Erbitux, C225) and any growth factor or growth factor receptor antibodies disclosed by Stern et al. Critical reviews in oncology/haematology, 2005, Vol. 54, ppl 1-29). Such inhibitors also include tyrosine kinase inhibitors such as inhibitors of the epidermal growth factor family (eg EGFR family tyrosine kinase inhibitors such as N-(3-chloro-4-fluorophenyl)-7-methoxy-6-(3-morpholinopropoxy) quinazolin-4-amine (gefitinib, ZD1 839), N-(3-ethynylphenyl)-6,7-bis(2-methoxyethoxy) quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (eg Ras/Raf signalling inhibitors such as farnesyl transferase inhibitors including sorafenib (BAY 43-9006), tipifarnib (R1 15777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; Aurora kinase inhibitors (eg AZD1152, PH739358, VX-680, MLN8054, R763, MP235, MP529, VX-528 and AX39459) and cyclin dependent kinase inhibitors such as CDK2 and/or CDK9 inhibitors;
anti-angiogenic agents such as those which inhibit the effects of vascular endothelial growth factor (eg the anti-vascular endothelial cell growth factor antibody bevacizumab (Avastin™) and VEGF receptor tyrosine kinase inhibitors such as vandetanib (ZD6474), vatalanib (PTK787), sunitinib (SU1 1248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within International Patent Publication No WO 00/47212), compounds such as those disclosed in International Patent Publication Nos WO97/22596, WO 97/30035, WO 97/32856 and WO 98/13354, and compounds that work by other mechanisms (eg linomide, inhibitors of integrin αvβ3 function and angiostatin);
vascular damaging agents such as Combretastatin A4 and compounds disclosed in International Patent Publication Nos WO 99/02166, WO 00/40529, WO 00/41669, WO 01/92224, WO 02/04434 and WO 02/08213;
an endothelin receptor antagonist such as zibotentan (ZD4054) or atrasentan;
antisense therapies such as those which are directed to the targets listed above, such as ISIS 2503, an anti-ras antisense;
gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Where used in combination with other anti-cancer agents, the Rilpivirine (or an analogue thereof) and the other anti-cancer agent may be administered in the same pharmaceutical composition or in separate pharmaceutical compositions. If administered in separate pharmaceutical compositions, the Rilpivirine (or an analogue thereof) and the other anti-cancer agent may be administered simultaneously or sequentially in any order (eg within seconds or minutes or even hours (eg 2 to 48 hours).

The methods, uses and composition of the present disclosure are typically applied to the treatment and/or prevention of cancer or another proliferative cell disease or condition in a human subject. However, the subject may also be selected from, for example, livestock animals (eg cows, horses, pigs, sheep and goats), companion animals (eg dogs and cats) and exotic animals (eg non-human primates, tigers, elephants etc).

Rilpivirine and the analogues thereof may be formulated into a pharmaceutical composition with a pharmaceutically acceptable carrier, diluent and/or excipient. Examples of suitable carriers and diluents are well known to those skilled in the art, and are described in, for example, Remington’s Pharmaceutical Sciences, Mack Publishing Co., Easton, PA 1995. Examples of suitable excipients for the various different forms of pharmaceutical compositions described herein may be found in the Handbook of Pharmaceutical Excipients, 2^{nd} Edition, (1994), Edited by A Wade and PJ Weller. Examples of suitable carriers include lactose, starch, glucose, methyl cellulose, magnesium stearate, mannitol, sorbitol and the like. Examples of suitable diluents include ethanol, glycerol and water. The choice of carrier, diluent and/or excipient may be made with regard to the intended route of administration and standard pharmaceutical practice.

A pharmaceutical composition comprising Rilpivirine (or an analogue thereof) may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, dyes and even flavouring agents may be provided in the pharmaceutical composition. Examples of preservatives include sodium benzoate, sorbic acid and esters of p-hydroxybenzoic acid. Anti-oxidants and suspending agents may be also used.

A pharmaceutical composition comprising Rilpivirine (or an analogue thereof) may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration. For oral administration, particular use may be made of compressed tablets, pills, tablets, gellules, drops, and capsules. For other forms of administration, a pharmaceutical composition may comprise solutions or emulsions which may be injected intravenously, intraarterially, intrathecally, subcutaneously, intradermally, intraperitoneally or intramuscularly, and which are prepared from sterile or sterilisable solutions. A pharmaceutical composition comprising Rilpivirine (or an analogue thereof) may also be in form of suppositories, pessaries, suspensions, emulsions, lotions, ointments, creams, gels, sprays, solutions or dusting powders. A pharmaceutical composition may be formulated in unit dosage form (ie in the form of discrete portions containing a unit dose, or a multiple or sub-unit of a unit dose).

Rilpivirine and analogues thereof may be prepared by, for example, the general synthetic methodologies described in International Patent Publication No WO 03/016306.

The methods, uses and composition of the present disclosure are hereinafter further described with reference to the following, non-limiting examples.

### EXAMPLES

### Example 1

### Methods and Materials

### Cell Viability Assays (MTT and Resazurin Assays)

The MTT (3-(4,5-dimethylthiazol-2-yl)-2,5-diphenyl tetrazolium bromide; Sigma-Aldrich, St Louis, MO, United States of America) assay was used to determine half maximal inhibition values (IC₅₀) in response to drug treatment using a number of different adherent cancer cell lines. The MTT was converted by metabolically active cells into an insoluble formazan product which was solubilised with 100% DMSO. The solubilised formazan gives absorbance at 560 nM which is directly proportional to the number of viable cells. The assay was carried out as previously described (Wang S et al., J Med Chem 47(7):1662-1675, 2004). The absorbance of treated wells was read using an EnVision multi-label plate reader. The resazurin assay was carried out for non-adherent cell lines as reported previously (Yu M et al., EJMC 95:116-126, 2015). IC₅₀ values were determined by non-linear regression analysis using GraphPad Prism software.

### Kinase Assay

The ability of compounds to inhibit kinases was measured using ADP-Glo^{Tm} assay kits (Promega, Madison, WI, United States of America) or externally using radioisotope based assays (Reaction Biology Corporation, Malvern, PA, United States of America and Eurofins discovery services, France). The ADP-Glo assay was performed as reported previously (Basnet SK et al., Mol Pharmacol 88(5):935-948, 2015).The assay plate was read for luminescence using Envision multi-label plate reader (PerkinElmer, Waltham, MA, United States of America). Half-maximal inhibition (IC₅₀) values were determined from a plot of percent residual activity versus concentration of test compounds using GraphPad Prism software. Kᵢ values were calculated from IC₅₀ values using the Cheng-Prusoff equation: Kᵢ= IC₅₀/ [1 + ([ATP]/Km ATP)], where [ATP] is the ATP concentration used for the IC₅₀ determination and Km ATP for each kinase is determined from an individual experiment.

### Cell Cycle Analysis

The effect of Rilpivirine on cell cycle distribution in different cancer cell lines was evaluated by flow cytometric analysis. Briefly, cells were seeded in 6-well plates at a density of 6 × 10⁴ cells/well. All plates were incubated overnight at 37° C. in a 5% CO₂ incubator. Following the incubation, the test compounds (Rilpivirine (Ril) and comparators, Alisertib (Ali; a known Aurora A inhibitor) and Etravirine (Etra; 4-[[6-amino-5-bromo-2-[(4-cyanophenyl)amino]-4-pyrimidinyl]oxy]-3,5-dimethylbenzonitrile - a structurally similar NNRTI drug compound sold under the brand name Intelence® (Janssen Therapeutics)) were added to individual wells and the plates were incubated at same condition for 24 h, 48 h or 72 h. Subsequently, the medium was removed from the wells and transferred into fluorescence-activated cell sorting (FACS) tubes. The adherent cells remaining were trypsinised, re-suspended in media and transferred into the FACS tubes. Cells were centrifuged, fixed with cold 70% ethanol for 15 min and pelleted. The pelleted cells were then re-suspended in 200 µL propidium iodide (PI) solution (50 µg/mL propidium iodide, 0.1 mg/mL ribonuclease A, 0.1% sodium citrate, 0.1% triton X-100) and incubated in the dark for 1.5 h at room temperature. Following the incubation, 200 µL PBS was added. Samples were assessed using a flow cytometer (Cytoflex; Beckman Coulter Inc, Brea, CA, United States of America) and data were analysed using the Cytexpert software.

### Apoptosis Analysis

Induction of apoptosis was examined using Annexin-V FITC/PI stains and a similar experimental procedure for cell cycle was followed apart from the apoptosis analysis which did not require fixation of cells with 70% ethanol. Instead of ethanol fixation, the cell number of each sample was counted, and the cells were diluted to 1×10⁵ cells with 1 mL PBS in fresh FACS tubes. Cells were centrifuged and then re-suspended in 1 mL of cold PBS (whole step repeated twice). Pelleted cells were resuspended in 100 µL binding buffer, and subsequently 3 µL of Annexin V and 3 µL of PI were added to all tubes and incubated in the dark for 15 min at room temperature. Following the incubation period, 200 µL of 1 x binding buffer was added. Samples were assessed using a flow cytometer (Cytoflex) within 1 h of staining and data were analysed using Cytexpert software.

### Western Blot Analysis

For Western blotting, cells (8×10⁵) were seeded in a culture dish with 10 mL medium and incubated overnight at 37° C., 5% CO₂. Untreated and compound-treated cells were lysed using phosphate lysis buffer and protease inhibitors. The protein concentration of each sample was determined by Bio-Rad DC™ protein assay (Bio-Rad Laboratories, Hercules, CA, United States of America). The protein was deactivated at 95° C. for 5 min and then resolved on 4-20% polyacrylamide gels by electrophoresis. Proteins were transferred to polyvinylidene difluoride (PDVF) membrane and blocked for 1 h with 10% skimmed milk (SM) in Tris-buffered saline and Tween (TBST). After adding primary antibody, the membranes were incubated overnight on a rocker in a 4° C. cold room. The following day, membranes were washed in TBST (4×20 min), incubated for at least 1 h at room temperature with the appropriate horseradish peroxidase conjugated secondary antibody. Following this, the blots were washed with TBST (4×20 min) again. The blots were then treated with Western blotting detection reagent and the band intensity was determined using a Bio-Rad ChemiDoc™ MP imaging system (Bio-Rad Laboratories). All the antibodies used for protein detection were from Cell Signaling Technology (Danvers, MA, United States of America). The primary and secondary antibodies used were obtained from Cell Signalling Technology (Danvers, MA, United States of America).

### Colony Formation Assay

A total of 1000-1500 cells/well were plated on a 6 well plate and allowed to adhere for 6-8 hours. Appropriate concentrations of Rilpivirine were added and then the cells were put back into the incubator for 7-14 days. The medium was changed every 3 days. After the incubation, the cells were washed with 1 ml PBS carefully. Cells were then fixed with 1 % Formaldehyde and stained with crystal violet. Colonies were counted with >50 cells by eye and images were taken using a camera fitted with microscope.

### RT-qPCR (Quantitative Reverse Transcription Polymerase Chain Reaction)

The expression of different genes at the mRNA level was assayed by RT-qPCR. Briefly, cells (1×10⁶) were seeded in 10 cm tissue culture plates and treated with compounds after having been cultured overnight at 37° C., 5% CO₂. RNA was extracted using the High Pure RNA Isolation Kit (Roche Applied Science, Castle Hill, NSW, Australia) according to the manufacturer’s instructions. The concentration of RNA in each sample was determined using a NanoDrop® ND1000 spectrophotometer (ThermoFisher Scientific, Waltham, MA, United States of America). 1 µg of RNA was used in a 20 µL reverse transcription reaction using a Transcriptor First Strand cDNA Synthesis Kit (Roche Applied Science). RT-qPCR was completed using duplicate samples with cDNA using SYBR Green I dye (Roche Applied Science) and performed using Light Cycler LC96 (Roche Applied Science, Penzberg, Germany). GAPDH mRNA was used as a reference sequence and data was analysed using Bio-Rad CFX manager version 3.1.

### Combination Data Analysis

Growth inhibition data was obtained by MTT cell viability assays. Then the synergy surface software Combenefit (Di Veroli GY et al., Bioinformatics 32(18):2866-2868, 2016) was used to identify synergistic drug combinations. The single agent inhibition value was used to calculate a drug combination surface under the assumption of additive effect. The Bliss mathematical model (Liu Q et al., Stat Biopharm Res 10(2):112-122, 2018) was applied to obtain this additive value. Regions of synergy were then identified by comparing obtained data from a combination with the calculated additive effect. This was done by subtracting the calculated additive inhibition values from the measured inhibition values to obtain the final difference values. Positive values indicate synergy and negative values indicate antagonism in the final synergy surface.

### Results

### Effect of Rilpivirine on Cancer Cell Proliferation

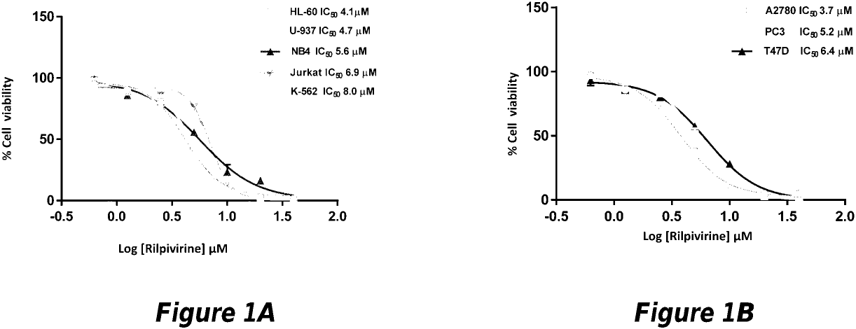

Using Resazurin or MTT assays, Rilpivirine was tested for its effect on cancer cell proliferation with a number of different cancer cell lines. The results are shown in FIGS. 1A-B. It was found that, for all cell lines, Rilpivirine inhibited cell proliferation with an IC₅₀ value ranging from 3 to 8 µM. The graphs shown in FIGS. 1A-B were calculated from one of three independent experiments performed in triplicates. Best-fit curves were determined using Prism software and were used to calculate the IC₅₀ values.

### Effect of Rilpivirine on Kinase Profiling

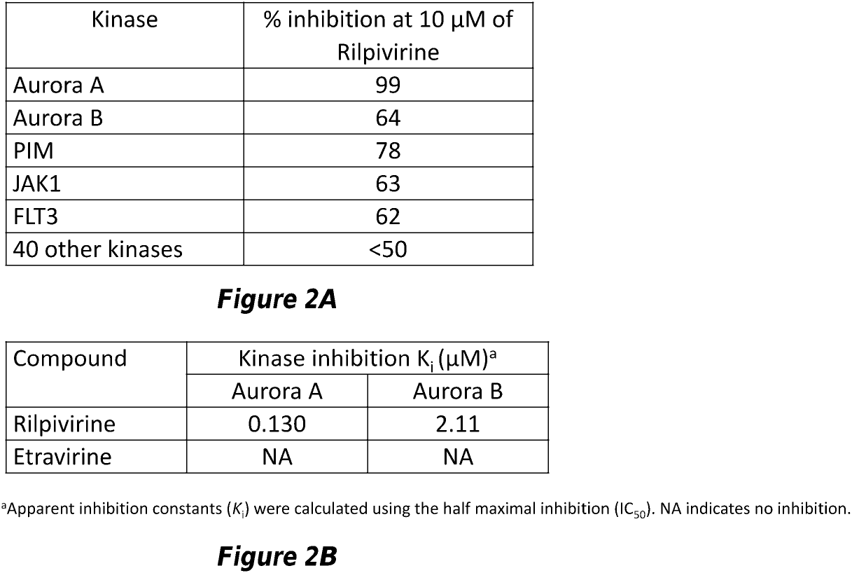

Rilpivirine was initially tested for inhibitory activity against a panel of 45 different kinase enzymes including, particularly, Aurora A, Aurora B, PIM1, JAK1 and FLT3. The related NNRTI, Etravirine, was also tested for inhibitory activity against Aurora A and Aurora B. The results of this kinase inhibition profiling are shown in the table forming FIG. 2. The results clearly showed that at a concentration of 10 µM, Rilpivirine potently inhibits the Aurora A kinase and also has some inhibitory activity against the Aurora B, PIM1, JAK1 and FLT3 kinases, whereas Etravirine showed no inhibitory activity against either of the Aurora kinases.

In a subsequent kinase profiling experiment, using an expanded panel of 429 different kinases, the inhibitory activity of Rilpivirine was confirmed using a reduced concentration of 1 µM. The results demonstrated that Rilpivirine is a selective inhibitor of Aurora A. Other than Aurora A, significant levels of inhibition were only observed against PIM1, and two other Src family tyrosine kinases (LYN and YES kinase). The percent inhibition against Aurora A, PIM1, LYN and YES kinases were 86, 78, 77 and 83% respectively at the concentration of 1 µM of Rilpivirine. The other 424 kinases were inhibited by less than 70 % at this concentration.

### Effect of Rilpivirine on Cell Cycle Arrest in P53 Null or Mutant Cell Lines

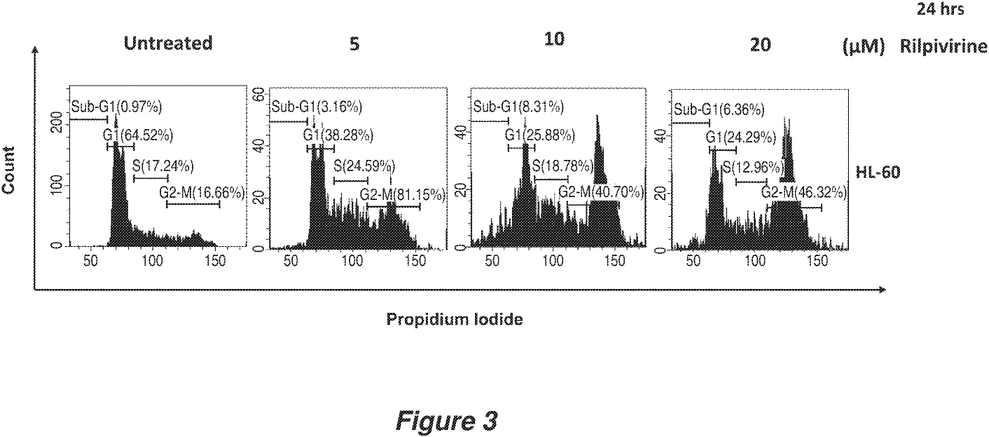

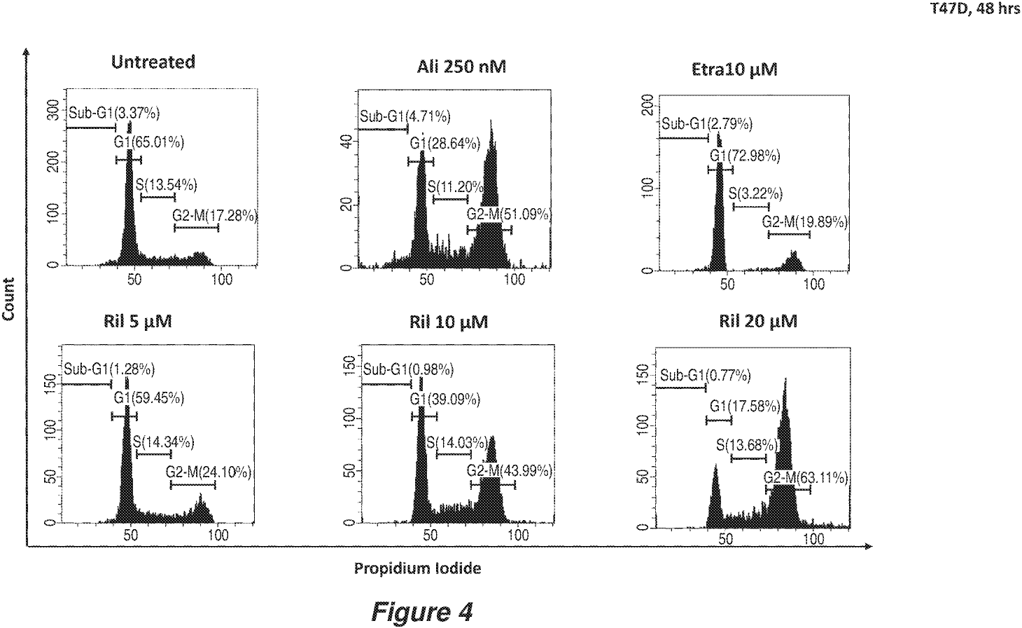

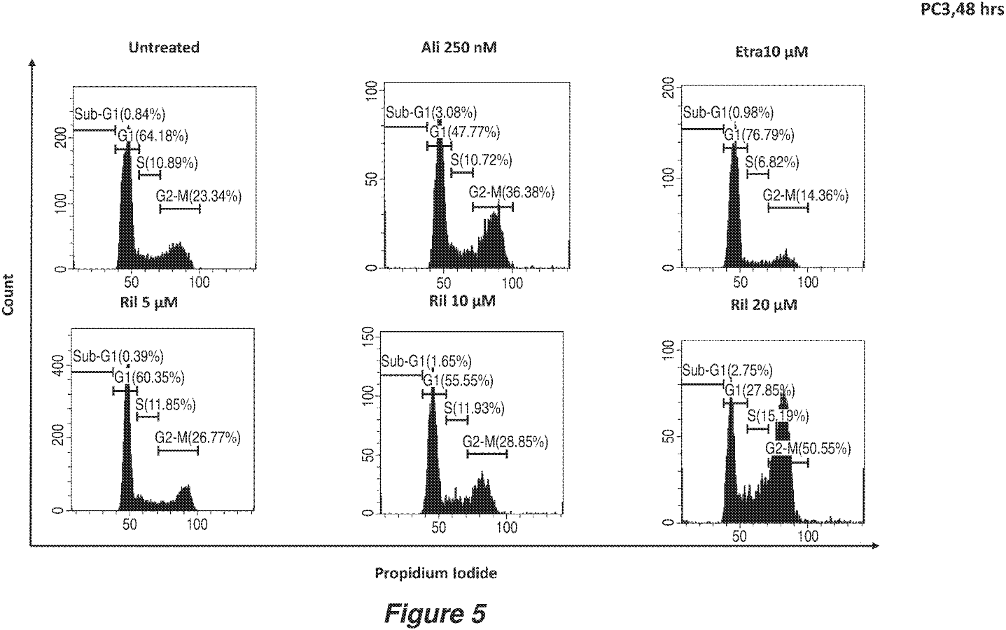

When HL-60 (leukaemia; p53 status “null”), T47D (breast; p53 status “mutant”) or PC3 (Prostate; p53 status “mutant”) cells were treated with Rilpivirine at a concentration range of 5, 10 and 20 µM for 24 hrs, the cell cycle analysis indicated that the cells were arrested in the G2/M Phase. FIG. 3 shows the G2/M arrest with Rilpivirine in HL-60 cells. It can be seen that the numbers of cells in G2/M approximately doubled in the presence of Rilpivirine. Evidence of endoreduplication is also apparent in the figure. Similar G2/M arrest was also evident in T47D (breast cancer) and PC3 (prostate) cells (see FIGS. 4 and 5). In contrast, the related NNRTI (Non-nucleoside Reverse Transcriptase Inhibitors), Etravirine, showed no G2/M arrest in T47D and PC3 cells (FIGS. 4 and 5).

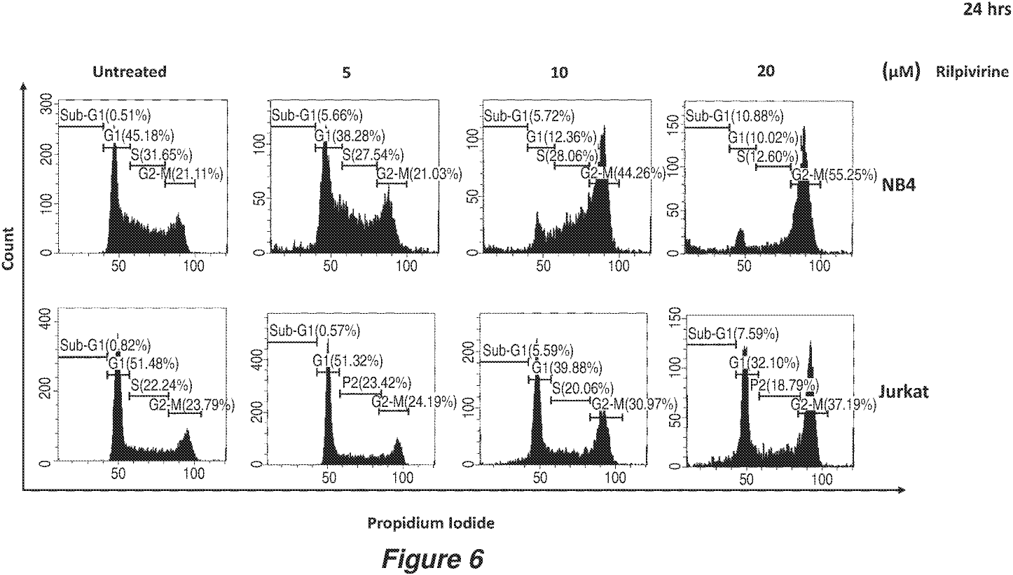

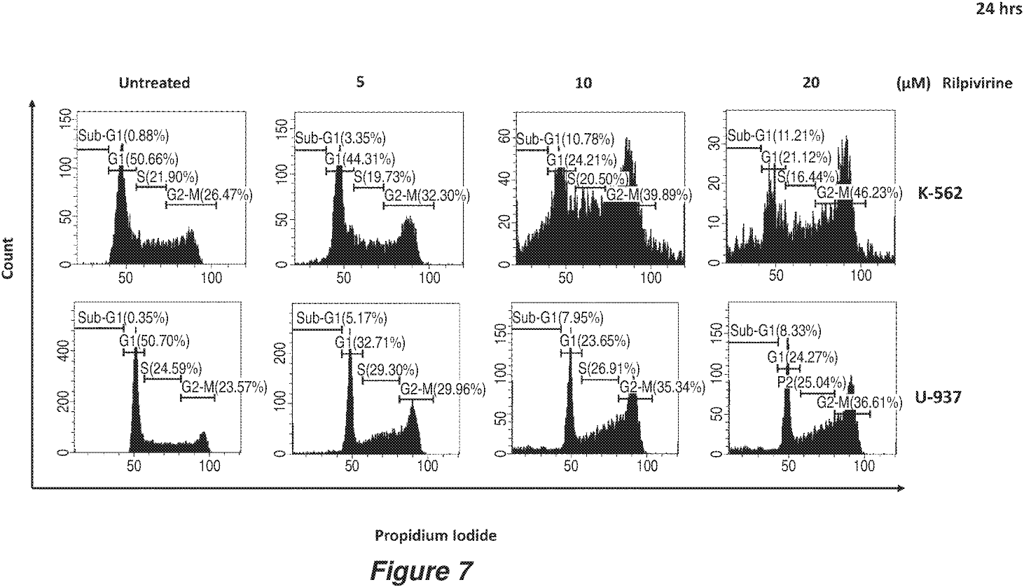

In Miapaca-2 (pancreatic) cancer cell lines, the pattern described above for Rilpivirine was repeated. In particular, after incubation with Rilpivirine for 48 hours, G2/M arrest increased with increasing concentrations of the drug (data not shown). Further, in experiments with the additional leukaemic cell lines NB4, Jurkat, K-562 and U-937 cells (all p53 mutant cell lines), treatment with Rilpivirine again showed that the cells were arrested in G2/M phases (FIGS. 6 and 7). And again, with a p53 mutant ovarian cancer cell line, SKOV-3, Rilpivirine treatment for 72 hours at various concentrations (ie 5 µM, 10 µM and 20 µM), caused G2/M arrest in a dose-dependent manner (data not shown).

### Effect of Rilpivirine on Apoptosis in Cancer Cell Lines

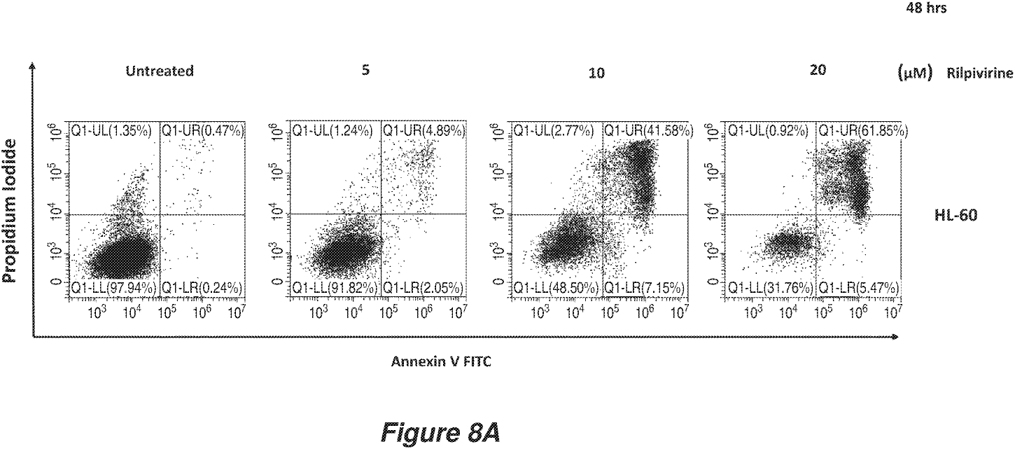

The results of experiments designed to assess the effect of Rilpivirine on cellular apoptosis are shown in FIGS. 8A-B. It can be seen from these figures that when HL-60, NB4 and Jurkat cells were treated with increasing concentration of Rilpivirine, there is an increase in the number of apoptotic cells (Lower and Upper Right quadrants). Similar results were achieved with Rilpivirine treatment of K-562 and U-937 cells. Note the effectiveness of Rilpivirine in HL-60 and NB4 cells on apoptosis were more pronounced which is consistent with the G2/M cell cycle arrest for these two cell lines. Moreover, it was also observed that with increasing concentrations of Rilpivirine, there is an appearance in the cell lysates of cleaved PARP (poly (ADP-ribose) polymerase) and downregulation of bc1-2 indicating apoptosis (data not shown).

### Effect of Rilpivirine on Aurora A Auto-phosphorylation and Clonogenic Survival

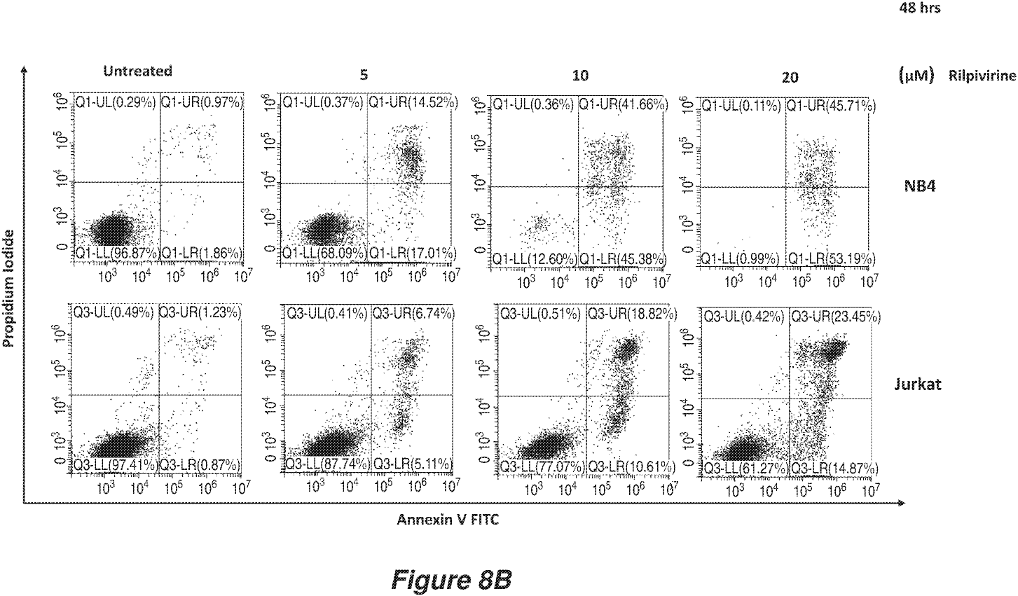

Experiments were conducted to determine the effect of Rilpivirine (24 hour treatment of HL-60 and NB4 cells at concentrations of 5 µM, 10 µM and 20 µM) on the level of auto-phosphorylation of Aurora A at Thr²⁸⁸residue. FIG. 9 shows Western blot of phosphorylated Aurora A^{T288} (detected using Phospho-Aurora A(Thr 288) antibody, Cell Signalling Technology) capable of specifically detecting the phosphorylated form) and total Aurora A (detected using Aurora A antibody, Cell Signalling Technology). It was found that with an increasing concentration of Rilpivirine, there is a marked decrease in the level of auto-phosphorylated protein; thereby indicating that Rilpivirine inhibits phosphorylation of Aurora A. Using p53 wild-type (WT) A2780 cells, a similar decrease in phosphorylated Aurora-A protein was observed with increasing concentrations of Rilpivirine (data not shown). In addition, Rilpivirine shows a clear inhibition of clonogenic survival in A2780 cells (data not shown). Similar effects were seen on clonogenic survival with p53 wild type M-249 (melanoma) cells, p53 mutant T47D (breast) and PC3 (prostate) cells.

### Effect of Rilpivirine on Cell Cycle Arrest in P53 Wild Type Cell Lines

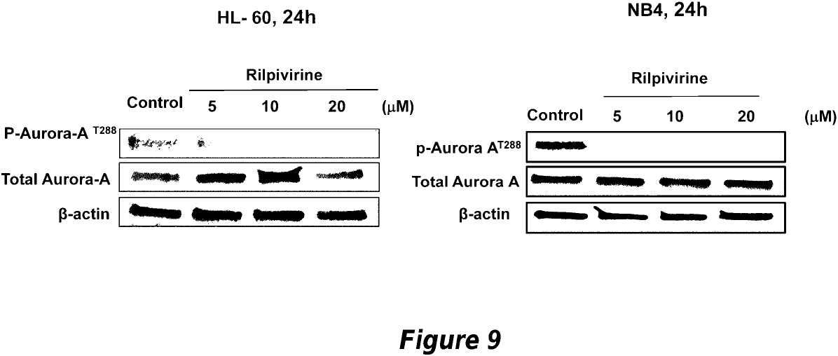

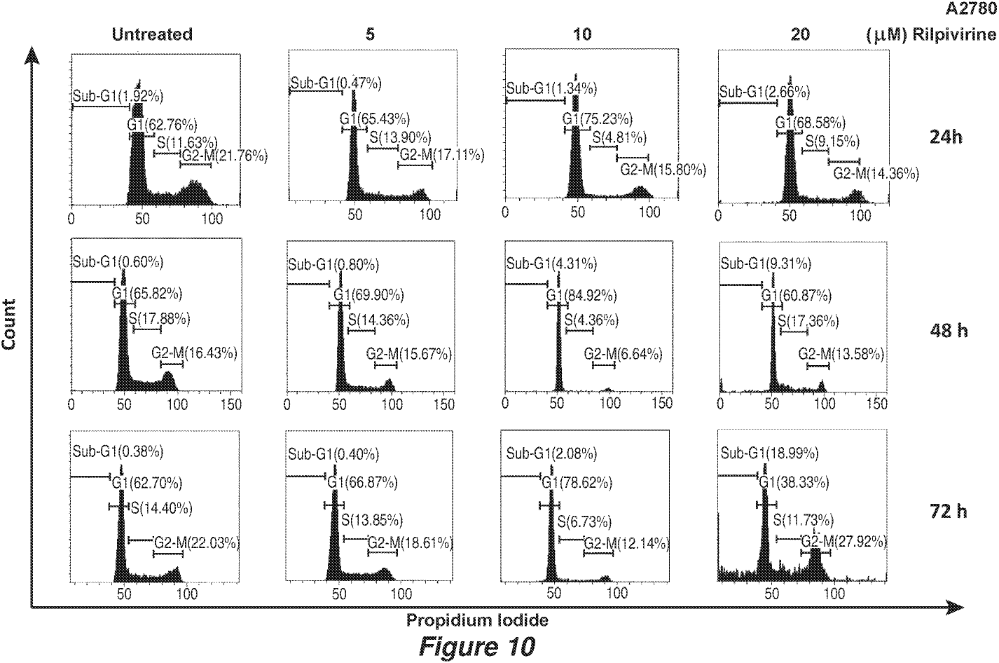

When p53 wild type cells (ie cells with functional p53) were treated with Rilpivirine for 24, 48 and 72 hours of incubation, it was surprisingly found from cell cycle analysis that the cells were arrested in the G1 phase (for all time points with a concentration of 10 µM of Rilpivirine) or accumulated in the sub-G1 phase (for treatments with Rilpivirine concentrations greater than 10 µM). FIG. 10 shows the G1 arrest with Rilpivirine in p53 WT ovarian A2780 cells. FIG. 11 shows similar G1 arrest results with the p53 WT ovarian OVCAR-5 cell line.

### Discussion

The examples showed that Rilpivirine induces cell cycle arrest in cancer cells regardless of their p53 status. However, in the p53 negative cells tested (ie HL-60, NB4, Jurkat, K562, U-937, T47D, Miapaca-2, PC-3 and SKOV-3), cell cycle arrest was in the G2/M phase (as was also seen for the comparator Aurora A inhibitor, Altiserb), whereas in p53 wild type ovarian cells (ie A2780 and OVCAR-5), Rilpivirine induced G1 cell cycle arrest. It was also observed that Rilpivirine was capable of inducing apoptosis and inhibiting clonogenic survival in cells independent of p53 status.

### Example 2

### Methods and Materials

Methods and materials used in the further experimentation described in this example were as described in Example 1.

### Results

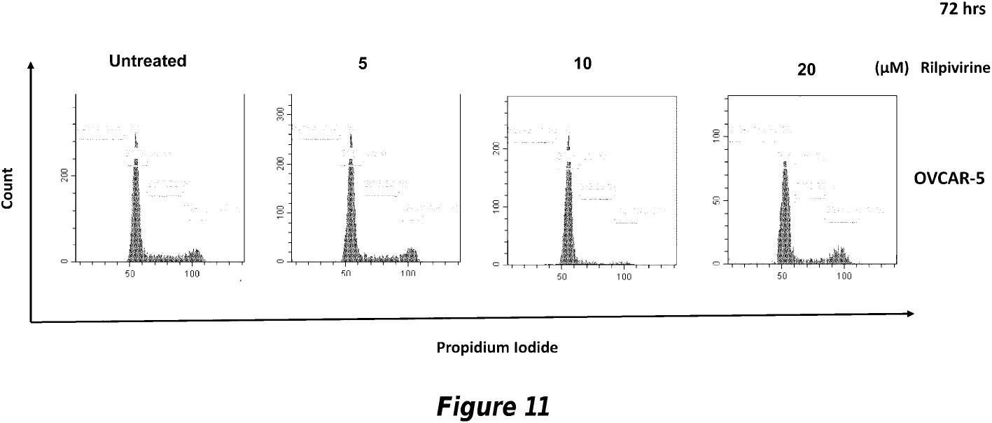

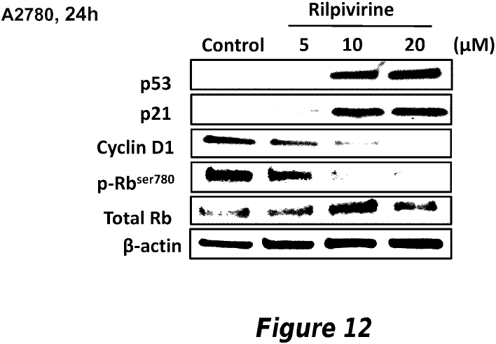

Effect of Rilpivirine on cell cycle regulatory proteins in p53 wild type cell lines In the previous example, it was found that when Rilpivirine was used to treat p53 wild type cells (ie cells with functional p53), the cells were induced to predominantly arrest in the G1 phase. Here, an examination was made on the effect of Rilpivirine on various regulatory proteins involved in G1-S phase transition. Consistent with the known properties of G1 arrest, Rilpivirine was found to decrease the phosphorylation of retinoblastoma (Rb) protein and cyclin D1 (see FIG. 12). The effect of Rilpivirine on cyclin D1 was also confirmed by measuring cyclin D1 messenger RNA (mRNA) following treatment of A2780 cells with Rilpivirine for 24 hours (FIG. 13); a decrease in cyclin D1mRNA was observed in a dose-dependent manner.

In addition, Rilpivirine also increased the cellular levels of the p53 and p21 proteins (see FIG. 12). In the case of p53 (p21 not tested), analysis of p53 mRNA also showed that Rilpivirine upregulated p53 expression with increasing Rilpivirine concentration following 24 hours treatment (FIG. 13). At 20 µM, Rilpivirine caused a dramatic increase in p53 expression.

### Effect of Rilpivirine on Apoptosis in P53 Wild Type Cell Lines

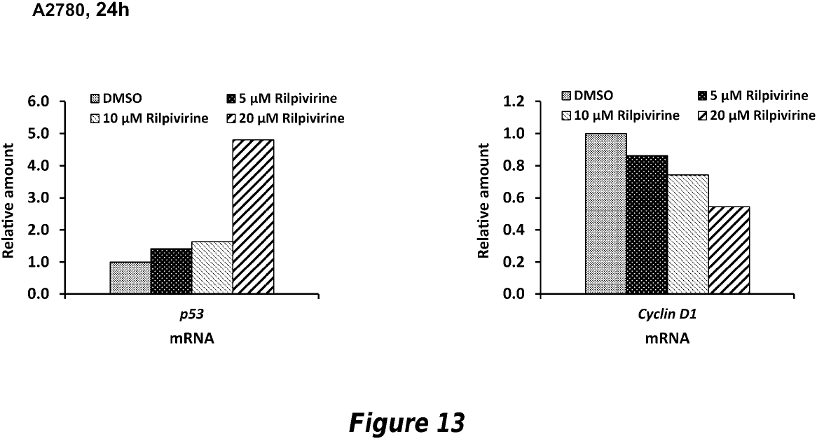

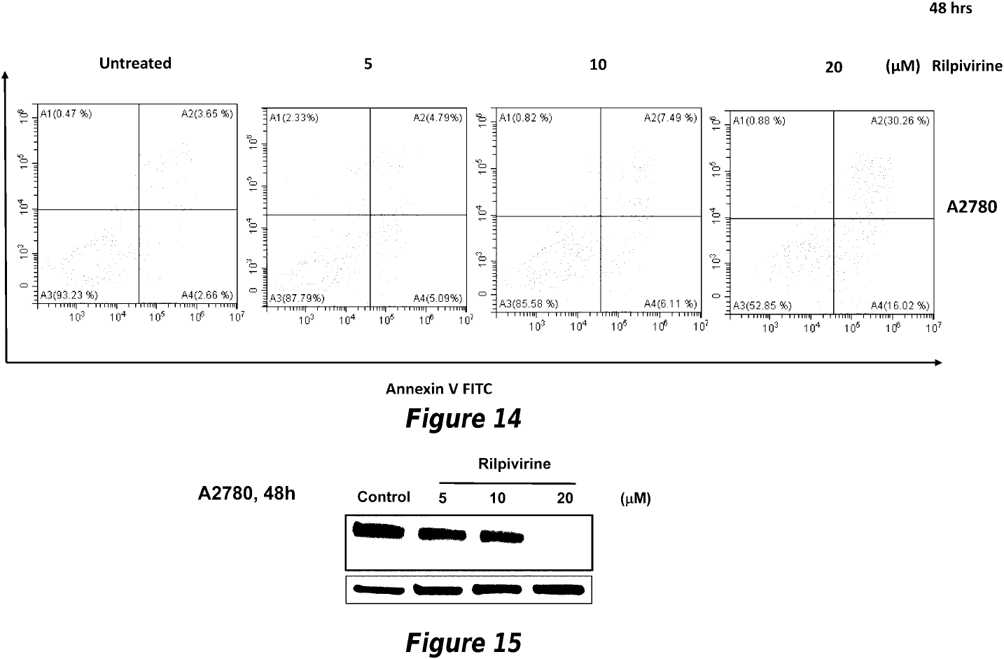

Further experimentation was conducted to assess whether Rilpivirine could induce apoptosis in cells with functional p53. The results are shown in FIG. 14. Flow cytometric analysis of A2780 cells treated for 48 hours with increasing concentrations of Rilpivirine clearly showed an increasing population of apoptotic cells. It was also found that this is associated with the presence of cleaved PARP (FIG. 15) indicating apoptosis.

### Effect of Rilpivirine in Combination With Docetaxel

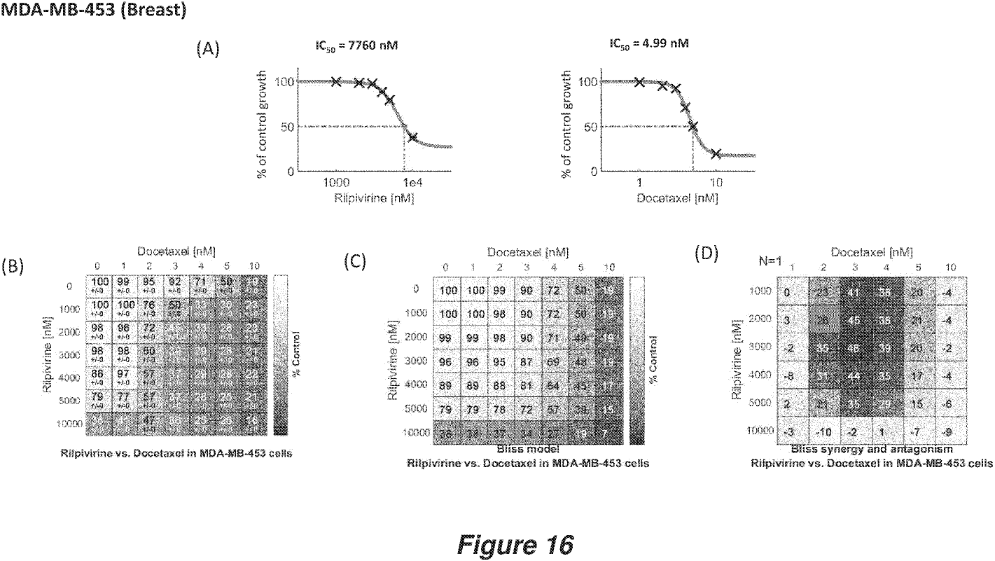

Further experimentation was conducted to determine the efficacy of Rilpivirine in combination with existing chemotherapy such as taxanes (eg paclitaxel and docetaxel) in breast and ovarian cancer cell lines. The MTT assay was used for investigating concentration ranges of 1-10 nM Docetaxel and 1000-10000 nM Rilpivirine at 72 h to obtain the growth inhibition data. Then the synergy surface software Combenefit and the mathematical model Bliss was applied to identify the regions of synergy. FIG. 16 shows the effect of Rilpivirine in combination with Docetaxel. A region of synergy was identified between Rilpivirine 1000 nM to 5000 nM and Docetaxel 2 nM to 4 nM, with the strongest synergy seen at Rilpivirine 3000 nM and Docetaxel 3 nM which is evidenced by a positive synergy score (Synergy score of > 25 potentially significant). Antagonism was seen with the higher concentrations of Docetaxel (> 4 nM) and Rilpivirine (> 5000 nM). A similar type of synergistic regions was found (data not shown) in breast cancer cell lines MDA-MB-468, MDA-MB-231, MCF-7 and ovarian cancer cell line A2780 in which regions of synergy were mostly identified between 2 to 3 nM Docetaxel and 3000 to 5000 nM Rilpivirine, but areas of antagonism were identified at higher concentrations of both drugs.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the methods and uses of Rilpivirine (and analogues thereof) and composition disclosed herein are not restricted by the particular application(s) described. Neither are the methods, uses and composition restricted in their preferred embodiment(s) with regard to the particular elements and/or features described or depicted herein. It will also be appreciated that the methods and uses of Rilpivirine (and analogues thereof) and composition disclosed herein are not limited to the embodiment or embodiments disclosed, but are capable of numerous rearrangements, modifications and substitutions without departing from the scope of the disclosure as set forth and defined by the following claims.

1. A method of treating and/or preventing cancer or another proliferative cell disease or condition in a subject, the method comprising administering to said subject a therapeutically effective amount of Rilpivirine or an analogue thereof, or a pharmaceutically acceptable salt, solvate or prodrug thereof, optionally in combination with a pharmaceutically acceptable carrier, diluent and/or excipient.

2. The method of claim 1, wherein the analogue is according to the following formula (I): 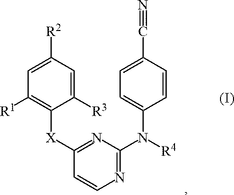
wherein:
R¹, R³ and R⁴ are each independently selected from the group consisting of H, saturated and unsaturated, optionally substituted, aliphatic hydrocarbons such as alkyl (eg a C₁₋₆ alkyl or, preferably, a C₁₋₃ alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne), halogen (especially Br or F), NO₂, CF₃, OH, optionally substituted O-alkyl (eg an O-C₁₋₆ alkyl, preferably, an O-C₁₋₃ alkyl such as O-CH₃), NH₂, optionally substituted NH-alkyl (eg a NH-C₁₋₆ alkyl, preferably, a NH-C₁₋₃ alkyl such as NH-CH₃), N(alkyl)₂ (such as N(CH₃)₂), and optionally substituted SH-alkyl (eg a SH-C₁₋₆ alkyl or, preferably, a SH-C₁₋₃ alkyl such as SHCH₃ and SHC(CH₃)); and
R² is selected from the group consisting of saturated and unsaturated aliphatic hydrocarbons such as alkyl (eg a C₁₋₆ alkyl or, preferably, a C₁₋₃ alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne) substituted with one or more substituents each independently selected from CN, NR⁵R^{6,} —C(═O)—NR⁵R⁶ and —C(═O)—C₁₋₆ alkyl; wherein R⁵ and R⁶ are each independently selected from H, OH, C₁₋₆ alkyl (preferably, a C₁₋₃ alkyl such as methyl), C₁₋₆ alkyloxy (preferably, a C₁₋₃ alkyloxy), C₁₋₆ alkylcarbonyl (preferably, a C₁₋₃ alkylcarbonyl), C₁₋₆ alkyloxycarbonyl (preferably, a C₁₋₃ alkyloxycarbonyl), NH₂, and mono- or di-(C₁₋₆ alkyl)amino; and
X is selected from NH, N(C₁₋₃ alkyl), O, S, CH₂ and C(CH₃)₂; or a pharmaceutically acceptable salt, solvate or prodrug thereof.

3. The method of claim 1 or 2, when used to treat and/or prevent cancer selected from leukaemias and ovarian, lung, colorectal, cervical, neural, breast, prostate and melanoma cancers.

4. The method of claim 3, wherein the cancer is characterised by kinase dysregulation.

5. The method of claim 4, wherein the cancer is characterised by dysregulation of one or more of Aurora A, Aurora B, PIM1 (Proto-oncogene serine/threonine-protein kinase), JAK1 (Janus kinase 1) FLT3 (fms like tyrosine kinase 3), YES and LYN ( Src family of tyrosine kinases) kinases .

6. The method of claim 3, wherein the cancer is characterised by Aurora A dysregulation.

7. The method of any one of claims 1 to 6, wherein the Rilpivirine or analogue thereof is administered in combination with one or more additional agent(s) for the treatment of cancer or another proliferative disease or condition.

8. The method of claim 7, wherein the additional agent(s) are selected from taxanes.

9. The method of claim 8, wherein the additional agent(s) is selected from docetaxel and paclitaxel.

10. The use of Rilpivirine or an analogue thereof, or a pharmaceutically acceptable salt, solvate or prodrug thereof, for treating and/or preventing cancer or another proliferative cell disease or condition.

11. The use of Rilpivirine or an analogue thereof, or a pharmaceutically acceptable salt, solvate or prodrug thereof, in the manufacture of a medicament for treating and/or preventing cancer or another proliferative cell disease or condition.

12. The use of claim 10 or 11, wherein the analogue is according to the following formula (I): 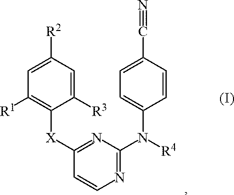
wherein:
R¹, R³ and R⁴ are each independently selected from the group consisting of H, saturated and unsaturated, optionally substituted, aliphatic hydrocarbons such as alkyl (eg a C₁₋₆ alkyl or, preferably, a C₁₋₃ alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne), halogen (especially Br or F), NO₂, CF₃, OH, optionally substituted O-alkyl (eg an O—C,₁₋₆ alkyl, preferably, an O-C₁₋₃ alkyl such as O—CH₃), NH₂, optionally substituted NH-alkyl (eg a NH-C₁₋₆ alkyl, preferably, a NH-C₁₋₃ alkyl such as NH-CH₃), N(alkyl)₂ (such as N(CH₃)₂), and optionally substituted SH-alkyl (eg a SH-C₁₋₆ alkyl or, preferably, a SH-C₁₋₃ alkyl such as SHCH₃ and SHC(CH₃)); and
R² is selected from the group consisting of saturated and unsaturated aliphatic hydrocarbons such as alkyl (eg a C₁₋₆ alkyl or, preferably, a C₁₋₃ alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne) substituted with one or more substituents each independently selected from CN, NR⁵R^{6,} —C(═O)—NR⁵R⁶ and —C(═O)—C₁₋₆ alkyl; wherein R⁵ and R⁶ are each independently selected from H, OH, C₁₋₆ alkyl (preferably, a C₁₋₃ alkyl such as methyl), C₁₋₆ alkyloxy (preferably, a C₁₋₃ alkyloxy), C₁₋₆ alkylcarbonyl (preferably, a C₁₋₃ alkylcarbonyl), C₁₋₆ alkyloxycarbonyl (preferably, a C₁₋₃ alkyloxycarbonyl), NH₂, and mono- or di-(C₁₋₆ alkyl)amino; and
X is selected from NH, N(C₁₋₃ alkyl), O, S, CH₂ and C(CH₃)_{2;} or a pharmaceutically acceptable salt, solvate or prodrug thereof.

13. The use of any one of claims 10 to 12, wherein the cancer is selected from leukaemias and ovarian, lung, colorectal, cervical, neural, breast, prostate and melanoma cancers.

14. The use of claim 13, wherein the cancer is characterised by kinase dysregulation.

15. The use of claim 14, wherein the cancer is characterised by dysregulation of one or more of Aurora A, Aurora B, PIM1 (Proto-oncogene serine/threonine-protein kinase), JAK1 (Janus kinase 1), FLT3 (fms like tyrosine kinase 3), YES and LYN (Src family of tyrosine kinases).

16. The use of claim 13, wherein the cancer is characterised by Aurora A dysregulation.

17. A pharmaceutical composition comprising Rilpivirine or an analogue thereof, or a pharmaceutically acceptable salt, solvate or prodrug thereof, and a pharmaceutically acceptable carrier, diluent and/or excipient for use in treating and/or preventing cancer or another proliferative cell disease or condition.

18. The composition of claim 17, wherein the analogue is according to the following formula (I): 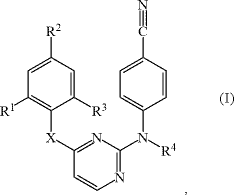
wherein:
R¹, R³ and R⁴ are each independently selected from the group consisting of H, saturated and unsaturated, optionally substituted, aliphatic hydrocarbons such as alkyl (eg a C₁₋₆ alkyl or, preferably, a C₁₋₃ alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne), halogen (especially Br or F), NO₂, CF₃, OH, optionally substituted O-alkyl (eg an O-C₁₋₆ alkyl, preferably, an O-C₁₋₃ alkyl such as O—CH₃), NH₂, optionally substituted NH-alkyl (eg a NH-C₁₋₆ alkyl, preferably, a NH-C₁₋₃ alkyl such as NH-CH₃), N(alkyl)₂ (such as N(CH₃)₂), and optionally substituted SH-alkyl (eg a SH-C₁₋₆ alkyl or, preferably, a SH-C₁₋₃ alkyl such as SHCH₃ and SHC(CH₃)); and
R² is selected from the group consisting of saturated and unsaturated aliphatic hydrocarbons such as alkyl (eg a C₁₋₆ alkyl or, preferably, a C₁₋₃ alkyl such as methyl), alkylene (eg a C₂₋₆ alkylene or, preferably, a C₂₋₃ alkylene such as ethylene) and alkyne (eg a C₂₋₆ alkyne or, preferably, a C₂₋₃ alkyne) substituted with one or more substituents each independently selected from CN, NR⁵R^{6,} —C(═O)—NR⁵R⁶ and —C(═O)—C₁₋₆ alkyl; wherein R⁵ and R⁶ are each independently selected from H, OH, C₁₋₆ alkyl (preferably, a C₁₋₃ alkyl such as methyl), C₁₋₆ alkyloxy (preferably, a C₁₋₃ alkyloxy), C₁₋₆ alkylcarbonyl (preferably, a C₁₋₃ alkylcarbonyl), C₁-₆ alkyloxycarbonyl (preferably, a C₁₋₃ alkyloxycarbonyl), NH₂, and mono- or di-(C₁₋₆ alkyl)amino; and
X is selected from NH, N(C₁₋₃ alkyl), O, S, CH₂ and C(CH₃)_{2;} or a pharmaceutically acceptable salt, solvate or prodrug thereof.

19. The composition of claim 17 or 18, wherein the cancer is selected from leukaemias and ovarian, lung, colorectal, cervical, neural, breast, prostate and melanoma cancers.

20. The composition of claim 19, wherein the cancer is characterised by kinase dysregulation.

21. The composition of claim 20, wherein the cancer is characterised by dysregulation of one or more of Aurora A, Aurora B, PIM (Proto-oncogene serine/threonine-protein kinase), JAK1 (Janus kinase 1) and FLT3 (fms like tyrosine kinase 3).

22. The composition of claim 19, wherein the cancer is characterised by Aurora A dysregulation.

23. The composition of any one of claims 17 to 22, further comprising one or more additional agent(s) for the treatment of cancer or another proliferative disease or condition.

24. The composition of claim 23, wherein the additional agent(s) are selected from taxanes.

25. The composition of claim 24, wherein the additional agent(s) is selected from docetaxel and paclitaxel.

26. A method for modulating Aurora A kinase activity in a cell, comprising introducing to or contacting said cell with an effective amount of Rilpivirine or an analogue thereof, or a pharmaceutically acceptable salt, solvate or prodrug thereof.
